(19) United States
(12) Patent Application Publication
(10) Pub. No.: US 2011/0275728 A1
(43) Pub. Date: Nov. 10, 2011

(54) DISPERSED TWO-COMPONENT POLYURETHANE FOAMS

(75) Inventors: Jan Schönberger, Solingen (DE); Sebastian Dörr, Dusseldorf (DE)

(73) Assignee: Bayer MaterialScience AG, Leverkusen (DE)

(21) Appl. No.: 13/145,000

(22) PCT Filed: Jan. 13, 2010
(86) PCT No.: PCT/EP2010/000121

§ 371 Date: Jul. 18, 2011

(30) Foreign Application Priority Data
Jan. 24, 2009 (EP) 09000992.9

(51) Int. Cl. C08L 75/08; C08L 75/12; A61L 15/26; C08J 9/30

(52) U.S. Cl. 521/70; 524/500; 524/537

(57) ABSTRACT

The invention relates to a method for the production of polyurethane foams, wherein a composition containing an anionic and a cationic polyurethane dispersion is expanded and dried.

The invention relates to a process for producing polyurethane foams wherein a composition containing an anionic polyurethane dispersion and a cationic polyurethane dispersion is frothed and dried.

The use of wound contact materials made of foams for treating weeping wounds is prior art. Owing to their high absorbency and their good mechanical properties, polyurethane foams produced by reaction of mixtures of diisocyanates and polyols or NCO-functional polyurethane prepolymers with water in the presence of certain catalysts and also (foam) additives are generally used. Aromatic diisocyanates are generally employed, since they are best foamable. Numerous forms of these processes are known, for example described in U.S. Pat No. 3,978,266, U.S. Pat No. 3,975,567 and EP-A 0 059 048. However, the aforementioned processes have the disadvantage that they require the use of reactive mixtures, containing diisocyanates or corresponding NCO-functional prepolymers, whose handling is technically inconvenient and costly, since appropriate protective measures are necessary for example.

One alternative to the above-described process, in which diisocyanates or NCO-functional polyurethane prepolymers are utilized, is a process based on polyurethane dispersions (which are essentially free of isocyanate groups) into which air is incorporated by vigorous stirring in the presence of suitable (foam) additives. So-called mechanical polyurethane foams are obtained after drying and curing. In connection with wound contact materials, such foams are described in EP-A 0 235 949 and EP-A 0 246 723, the foam either having a self-adherent polymer added to it, or being applied to a film of a self-adherent polymer. The examples recited in EP-A 0 235 949 and EP-A 0 246 723 additionally describe the obligatory use of polyaziridines as crosslinkers, which is no longer acceptable as we now know because of their toxicity. Crosslinking, moreover, requires high baking temperatures to be used; 100° C. to 170° C. are reported. U.S. Pat No. 4,655,210 describes the use of the aforementioned mechanical foams for wound dressings having a specific construction made up of backing, foam and skin contact layer. The foams produced by following the processes described in EP-A 0 235 949 and EP-A 0 246 723, moreover, have the immense disadvantage that the foams obtained are open-cell to a small degree only, as a result of which their absorbance of physiological saline and also their water vapour permeability are low.

Managing wounds of complex topology or covering particularly deep wounds is very difficult using ready-to-use, industrially manufactured, sheetlike wound contact materials since optimal covering of the wound surface is generally not accomplished, which retards the healing process. To achieve better coverage of deep wounds, EP-A-0 171 268 proposes using granules of microporous polyurethanes instead of compact wound dressings. Yet this does not provide optimal coverage of the wound either.

The application of a (flowable) composition that optimally conforms to the contours of the wound would eliminate the disadvantages of sheetlike wound contact materials. The two above-described processes, which each utilize either diisocyanates and/or NCO-functional polyurethane prepolymers or polyurethane dispersions in combination with polyaziridines to prepare the polyurethane foams, are not suitable for this, however: reactive compositions containing free isocyanate groups cannot be applied directly to the skin, even though this has been variously proposed (see for example WO 02/26848), for toxicological reasons. But even the use of polyurethane dispersions with polyaziridines as crosslinkers is as we now know not an option because the properties of the crosslinker are not generally recognized as safe by toxicologists.

A method of rapidly consolidating foamed polyurethane dispersions without thermal initiation is hitherto unknown, although the production of polyurethane films, i.e. non-porous materials, by coagulation with inorganic salts, for example, is a widely practiced technique in industry.

The present invention therefore has for its object to provide rapidly consolidating polyurethane foams, particularly for wound treatment, which are prepared using a composition that is free of isocyanate groups. The polyurethane foam shall in principle be obtainable also under ambient conditions, in that the polyurethane foams formed under these conditions shall have adequate mechanical properties within a very short time and without noticeable evolution of heat of reaction. The composition shall moreover be suitable for direct application to the skin, for example by spraying or pouring, in order that the wound may be optimally covered by the polyurethane foam. Optimal wound coverage, moreover, requires little if any volume shrinkage of the polyurethane foams during and after application.

It has now been found that, surprisingly, the combination of specific anionically hydrophilicized PU dispersions (I) and specific cationically hydrophilicized PU dispersions (II) provides foams which consolidate within a few seconds, without measurable evolution of heat.

Polyurethane foam wound contact materials within the meaning of the invention are porous materials, preferably having at least some open-cell content, which consist essentially of polyurethanes and protect wounds against germs and environmental influences in the sense of providing a sterile covering; ensure a suitable wound climate through suitable moisture permeability; and possess adequate mechanical strength.

The present invention accordingly provides a process for producing polyurethane foams wherein a composition obtainable by mixing of at least one aqueous anionically hydrophilicized polyurethane dispersion (I) and at least one cationically hydrophilicized PU dispersion (II) is during or after complete mixing of (I) and (II) frothed and cured.

Curing in relation to foam production is intended to be understood as meaning a consolidation of the included polymers, inter alia by drying, i.e. withdrawal of water and/or coagulation. Such a consolidated foam can therefore also be a foamed material which is still (water-)moist, yet is already solid in itself.

The invention further provides the composition containing the two dispersions (I) and (II), the polyurethane foams obtainable by following the process, and also their use, particularly their use as wound contact materials.

Preference is given to compositions prepared using anionically hydrophilicized polyurethane dispersions (I) having a —COO⁻ or —SO₃⁻ or —PO₃²⁻ group content of 2 to 500 milliequivalents per 100 g of solid resin and cationically hydrophilicized polyurethane dispersions (II) having a quaternary ammonium group content of 2 to 500 milliequivalents per 100 g of solid resin.

In addition to the dispersions (I) and (II), the compositions that are essential to the present invention may further comprise auxiliary and adjunct materials (III).

The aqueous anionically hydrophilicized polyurethane dispersions (I) in the compositions that are essential to the present invention are obtainable by
A) isocyanate-functional prepolymers being produced from
   A1) organic polyisocyanates
   A2) polymeric polyols having number average molecular weights in the range from 400 to 8000 g/mol, preferably in the range from 400 to 6000 g/mol and more preferably in the range from 600 to 3000 g/mol, and OH functionalities in the range from 1.5 to 6, preferably in the range from 1.8 to 3 and more preferably in the range from 1.9 to 2.1, and
   A3) optionally hydroxyl-functional compounds having molecular weights in the range from 62 to 399 g/mol and
   A4) optionally isocyanate-reactive, anionic or potentially anionic and/or optionally nonionic hydrophilicizing agents,
B) its free NCO groups then being wholly or partly reacted
   B1) optionally with amino-functional compounds having molecular weights in the range from 32 to 400 g/mol and
   B2) optionally with isocyanate-reactive, preferably amino-functional, anionic or potentially anionic hydrophilicizing agents
      by chain extension, and the prepolymers being dispersed in water before, during or after step B), any potentially anionic groups present being converted into the anionic form by partial or complete reaction with a neutralizing agent.

To achieve anionic hydrophilicization, A4) and/or B2) shall utilize hydrophilicizing agents that have at least one NCO-reactive group such as amino, hydroxyl or thiol groups and additionally have —COO⁻ or —SO₃⁻ or —PO₃²⁻ as anionic groups or their wholly or partly protonated acid forms as potentially anionic groups.

Preferred aqueous, anionic polyurethane dispersions (I) have a low degree of hydrophilic anionic groups, preferably from 2 to 200 milliequivalents and more preferably from less than 10 to 100 milliequivalents per 100 g of solid resin.

To achieve good sedimentation stability, the number average particle size of the polyurethane dispersions (I) is preferably less than 750 nm and more preferably less than 500 nm, determined by laser correlation spectroscopy.

The ratio of NCO groups of compounds of component A1) to NCO-reactive groups such as amino, hydroxyl or thiol groups of compounds of components A2) to A4) is in the range from 1.05 to 3.5, preferably in the range from 1.2 to 3.0 and more preferably in the range from 1.3 to 2.5 to prepare the NCO-functional prepolymer.

The amino-functional compounds in stage B) are used in such an amount that the equivalent ratio of isocyanate-reactive amino groups of these compounds to the free isocyanate groups of the prepolymer is in the range from 40 to 150%, preferably in the range from 50 to 125% and more preferably in the range from 60 to 100%.

Suitable polyisocyanates for component A1) include the well-known aromatic, araliphatic, aliphatic or cycloaliphatic polyisocyanates of an NCO functionality of ≧2.

Examples of such suitable polyisocyanates are 1,4-butylene diisocyanate, 1,6-hexamethylene diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or their mixtures of any desired isomer content, 1,4-cyclohexylene diisocyanate, 1,4-phenylene diisocyanate, 2,4- and/or 2,6-tolylene diisocyanate, 1,5-naphthylene diisocyanate, 2,2′- and/or 2,4′- and/or 4,4′-diphenylmethane diisocyanate, 1,3- and/or 1,4-bis(2-isocyanatoprop-2-yl)benzene (TMXDI), 1,3-bis(isocyanatomethyl)benzene (XDI) and also alkyl 2,6-diisocyanatohexanoates (lysine diisocyanates) having C1-C8-alkyl groups.

As well as the aforementioned polyisocyanates, it is also possible to use, proportionally, modified diisocyanates of uretdione, isocyanurate, urethane, allophanate, biuret, iminooxadiazinedione and/or oxadiazinetrione structure and also unmodified polyisocyanate having more than 2 NCO groups per molecule such as, for example, 4-isocyanatomethyl-1,8-octane diisocyanate (nonane triisocyanate) or triphenylmethane 4,4′,4″-triisocyanate.

Preferably, the polyisocyanates or polyisocyanate mixtures of the aforementioned kind have exclusively aliphatically and/or cycloaliphatically attached isocyanate groups and an average NCO functionality in the range from 2 to 4, preferably in the range from 2 to 2.6 and more preferably in the range from 2 to 2.4 for the mixture.

It is particularly preferable for A1) to utilize 1,6-hexamethylene diisocyanate, isophorone diisocyanate, the isomeric bis(4,4′-isocyanatocyclohexyl)methanes, and also mixtures thereof.

A2) utilizes polymeric polyols having a number average molecular weight Mₙ in the range from 400 to 8000 g/mol, preferably in the range from 400 to 6000 g/mol and more preferably in the range from 600 to 3000 g/mol. These preferably have an OH functionality in the range from 1.5 to 6, more preferably in the range from 1.8 to 3, most preferably in the range from 1.9 to 2.1.

Such polymeric polyols are the well-known polyurethane coating technology polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols and polyester polycarbonate polyols. These can be used in A2) individually or in any desired mixtures with one another.

Such polyester polyols are the well-known polycondensates formed from di- and also optionally tri- and tetraols and di- and also optionally tri- and tetracarboxylic acids or hydroxy carboxylic acids or lactones. Instead of the free polycarboxylic acids it is also possible to use the corresponding polycarboxylic anhydrides or corresponding polycarboxylic esters of lower alcohols for preparing the polyesters.

Examples of suitable diols are ethylene glycol, butylene glycol, diethylene glycol, triethylene glycol, polyalkylene glycols such as polyethylene glycol, also 1,2-propanediol, 1,3-propanediol, 1,3-butanediol, 1,4-butanediol, 1,6-hexanediol and isomers, neopentyl glycol or neopentyl glycol hydroxypivalate, of which 1,6-hexanediol and isomers, neopentyl glycol and neopentyl glycol hydroxypivalate are preferred. Besides these it is also possible to use polyols such as trimethylolpropane, glycerol, erythritol, pentaerythritol, trimethylolbenzene or trishydroxyethyl isocyanurate.

Useful dicarboxylic acids include phthalic acid, isophthalic acid, terephthalic acid, tetra-hydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethyl glutaric acid and/or 2,2-dimethylsuccinic acid. The corresponding anhydrides can also be used as a source of an acid.

When the average functionality of the polyol to be esterified is >2, monocarboxylic acids, such as benzoic acid and hexanecarboxylic acid can be used as well in addition.

Preferred acids are aliphatic or aromatic acids of the aforementioned kind. Adipic acid, isophthalic acid and, where appropriate, trimellitic acid are particularly preferred.

Hydroxy carboxylic acids useful as reaction participants in the preparation of a polyester polyol having terminal hydroxyl groups include for example hydroxycaproic acid, hydroxybutyric acid, hydroxydecanoic acid, hydroxystearic acid and the like. Suitable lactones include caprolactone, butyrolactone and homologues. Caprolactone is preferred.

A2) may likewise utilize hydroxyl-containing polycarbonates, preferably polycarbonate diols, having number average molecular weights Mₙ in the range from 400 to 8000 g/mol and preferably in the range from 600 to 3000 g/mol. These are obtainable by reaction of carbonic acid derivatives, such as diphenyl carbonate, dimethyl carbonate or phosgene, with polyols, preferably diols.

Examples of such diols are ethylene glycol, 1,2-propanediol, 1,3-propanediol, 1,3-butanediol, 1,4-butanediol, 1,6-hexanediol, 1,8-octanediol, neopentyl glycol, 1,4-bishydroxy-methylcyclohexane, 2-methyl-1,3-propanediol, 2,2,4-trimethyl-1,3-pentanediol, dipropylene glycol, polypropylene glycols, dibutylene glycol, polybutylene glycols, bisphenol A and lactone-modified diols of the aforementioned kind.

The polycarbonate diol preferably contains 40% to 100% by weight of hexanediol, preference being given to 1,6-hexanediol and/or hexanediol derivatives. Such hexanediol derivatives are based on hexanediol and have ester or ether groups as well as terminal OH groups. Such derivatives are obtainable by reaction of hexanediol with excess caprolactone or by etherification of hexanediol with itself to form di- or trihexylene glycol.

In lieu of or in addition to pure polycarbonate diols, polyether-polycarbonate diols can also be used in A2). The hydroxyl-containing polycarbonates preferably have a linear construction.

A2) may likewise utilize polyether polyols. Useful polyether polyols include for example the well-known polyurethane chemistry polytetramethylene glycol polyethers as are obtainable by polymerization of tetrahydrofuran by means of cationic ring opening.

Useful polyether polyols likewise include the well-known addition products of styrene oxide, ethylene oxide, propylene oxide, butylene oxides and/or epichlorohydrin onto di- or polyfunctional starter molecules. Polyether polyols based on the at least proportional addition of ethylene oxide onto di- or polyfunctional starter molecules can also be used as component A4) (nonionic hydrophilicizing agents).

Useful starter molecules include all prior art compounds, for example water, butyl diglycol, glycerol, diethylene glycol, trimethylolpropane, propylene glycol, sorbitol, ethylenediamine, triethanolamine, 1,4-butanediol. Preferred starter molecules are water, ethylene glycol, propylene glycol, 1,4-butanediol, diethylene glycol and butyl diglycol.

Particularly preferred embodiments of the polyurethane dispersions (I) contain as component A2) a mixture of polycarbonate polyols and polytetramethylene glycol polyols, the proportion of polycarbonate polyols in this mixture being in the range from 0% to 80% by weight and the proportion of polytetramethylene glycol polyols in this mixture being in the range from 100% to 20% by weight. Preference is given to a proportion of 50% to 100% by weight for polytetramethylene glycol polyols and to a proportion of 0% to 50% by weight for polycarbonate polyols. Particular preference is given to a proportion of 75% to 100% by weight for polytetramethylene glycol polyols and to a proportion of 0% to 25% by weight for polycarbonate polyols, each subject to the proviso that the sum total of the weight percentages for the polycarbonate and polytetramethylene glycol polyols is 100% and the proportion of component A2) which is contributed by the sum total of the polycarbonate and polytetramethylene glycol polyether polyols is at least 50% by weight, preferably 60% by weight and more preferably at least 70% by weight.

The compounds of component A3) have molecular weights in the range from 62 to 400 g/mol. A3) may utilize polyols of the specified molecular weight range with up to 20 carbon atoms, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrogenated bisphenol A, (2,2-bis(4-hydroxycyclohexyl)propane), trimethylolpropane, glycerol, pentaerythritol and also any desired mixtures thereof with each or one another.

Also suitable are ester diols of the specified molecular weight range such as α-hydroxybutyl-ε-hydroxycaproic acid ester, ω-hydroxyhexyl-γ-hydroxybutyric acid ester, β-hydroxyethyl adipate or bis(β-hydroxyethyl) terephthalate.

A3) may further utilize monofunctional, isocyanate-reactive, hydroxyl-containing compounds. Examples of such monofunctional compounds are ethanol, n-butanol, ethylene glycol monobutyl ether, diethylene glycol monomethyl ether, ethylene glycol monobutyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol. Compounds preferred for component A3) are 1,6-hexanediol, 1,4-butanediol, neopentyl glycol and trimethylolpropane.

An anionically or potentially anionically hydrophilicizing compound for component A4) is any compound which has at least one isocyanate-reactive group such as a hydroxyl group and also at least one functionality such as for example —COO⁻M⁺, —SO₃⁻M⁺, —PO(O⁻M⁺)₂ where M⁺ is for example a metal cation, H⁺, NH₄⁺, NHR₃⁺, where R in each occurrence may be C₁-C₁₂-alkyl, C₅-C₆-cycloalkyl and/or C₂-C₄-hydroxyalkyl, which functionality enters on interaction with aqueous media a pH-dependent dissociative equilibrium and thereby can have a negative or neutral charge. Useful anionically or potentially anionically hydrophilicizing compounds include mono- and dihydroxy carboxylic acids, mono- and dihydroxy sulphonic acids and also mono- and dihydroxy phosphonic acids and their salts. Examples of such anionic or potentially anionic hydrophilicizing agents are dimethylolpropionic acid, dimethylolbutyric acid, hydroxypivalic acid, malic acid, citric acid, glycolic acid, lactic acid and the propoxylated adduct formed from 2-butenediol and NaHSO₃ as described in DE-A 2 446 440, page 5-9, formula I-III. Preferred anionic or potentially anionic hydrophilicizing agents for component A4) are those of the aforementioned kind that have carboxylate or carboxyl groups and/or sulphonate groups.

Particularly preferred anionic or potentially anionic hydrophilicizing agents are those that contain carboxylate or carboxyl groups as ionic or potentially ionic groups, such as dimethylolpropionic acid, dimethylolbutyric acid and hydroxypivalic acid and salts thereof.

Useful nonionically hydrophilicizing compounds for component A4) include for example polyoxyalkylene ethers which contain at least one hydroxyl or amino group, preferably at least one hydroxyl group.

Examples are the monohydroxyl-functional polyalkylene oxide polyether alcohols containing on average 5 to 70 and preferably 7 to 55 ethylene oxide units per molecule and obtainable in a conventional manner by alkoxylation of suitable starter molecules (for example in Ullmanns Encyclopadie der technischen Chemie, 4th edition, volume 19, Verlag Chemie, Weinheim pages 31-38).

These are either pure polyethylene oxide ethers or mixed polyalkylene oxide ethers, containing at least 30 mol % and preferably at least 40 mol % of ethylene oxide units, based on all alkylene oxide units present.

Preferred polyethylene oxide ethers of the aforementioned kind are monofunctional mixed polyalkylene oxide polyethers having 40 to 100 mol % of ethylene oxide units and 0 to 60 mol % of propylene oxide units.

Preferred nonionically hydrophilicizing compounds for component A4) are those of the aforementioned kind, being block (co)polymers which are prepared by blockwise addition of alkylene oxides onto suitable starters.

Useful starter molecules for such nonionic hydrophilicizing agents include saturated monoalcohols such as methanol, ethanol, n-propanol, isopropanol, n-butanol, isobutanol, sec-butanol, the isomers pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, for example diethylene glycol monobutyl ether, unsaturated alcohols such as allyl alcohol, 1,1-dimethylallyl alcohol or oleic alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methylcyclo-hexylamine, N-ethylcyclohexylamine or dicyclohexylamine and also heterocyclic secondary amines such as morpholine, pyrrolidine, piperidine or 1H-pyrazole. Preferred starter molecules are saturated monoalcohols of the aforementioned kind. Particular preference is given to using diethylene glycol inonobutyl ether or n-butanol as starter molecules.

Useful alkylene oxides for the alkoxylation reaction are in particular ethylene oxide and propylene oxide, which can be used in any desired order or else in admixture in the alkoxylation reaction.

Component B1) may utilize di- or polyamines such as 1,2-ethylenediamine, 1,2-diaminopropane, 1,3-diaminopropane, 1,4-diaminobutane, 1,6-diaminohexane, isophoronediamine, isomeric mixture of 2,2,4- and 2,4,4-trimethylhexamethylenediamine, 2-methylpentamethylenediamine, diethylene-triamine, triaminononane, 1,3-xylylenediamine, 1,4-xylylenediamine, α,α,α′,α′-tetramethyl-1,3- and -1,4-xylylenediamine and 4,4-diaminodicyclohexylmethane and/or dimethylethylenediamine. It is also possible but less preferable to use hydrazine and also hydrazides such as adipodihydrazide.

Component B1) can further utilize compounds which as well as a primary amino group also have secondary amino groups or which as well as an amino group (primary or secondary) also have OH groups. Examples thereof are primary/secondary amines, such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine.

Component B1) can further utilize monofunctional isocyanate-reactive amine compounds, for example methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, or suitable substituted derivatives thereof, amide-amines formed from diprimary amines and monocarboxylic acids, monoketimes of diprimary amines, primary/tertiary amines, such as N,N-dimethylaminopropylamine.

Preferred compounds for component B1) are 1,2-ethylenediamine, 1,4-diaminobutane and isophoronediamine. Particular preference is given to using mixtures of the aforementioned diamines of component B1), particularly mixtures of 1,2-ethylenediamine and isophoronediamine and also mixtures of 1,4-diaminobutane and isophoronediamine.

An anionically or potentially anionically hydrophilicizing compound for component B2) is any compound which has at least one isocyanate-reactive group, preferably an amino group, and also at least one functionality such as for example —COO⁻M⁺, —SO₃⁻M⁺, —PO(O⁻M⁺)₂ where M⁺ is for example a metal cation, H⁺, NH₄⁺, NHR₃⁺, where R in each occurrence may be C₁-C₁₂-alkyl, C₅-C₆-cycloalkyl and/or C₂-C₄-hydroxyalkyl, which functionality enters on interaction with aqueous media a pH-dependent dissociative equilibrium and thereby can have a negative or neutral charge.

Useful anionically or potentially anionically hydrophilicizing compounds are mono- and diamino carboxylic acids, mono- and diamino sulphonic acids and also mono- and diamino phosphonic acids and their salts. Examples of such anionic or potentially anionic hydrophilicizing agents are N-(2-aminoethyl)-β-alanine, 2-(2-aminoethylamino)ethanesulphonic acid, ethylenediaminepropyl-sulphonic acid, ethylenediaminebutylsulphonic acid, 1,2- or 1,3-propylenediamine-β-ethyl-sulphonic acid, glycine, alanine, taurine, lysine, 3,5-diaminobenzoic acid and the addition product of IPDA and acrylic acid (EP-A 0 916 647, Example 1). It is further possible to use cyclohexyl-aminopropanesulphonic acid (CAPS) from WO-A 01/88006 as anionic or potentially anionic hydrophilicizing agent.

Preferred anionic or potentially anionic hydrophilicizing agents for component B2) are those of the aforementioned kind that have carboxylate or carboxyl groups and/or sulphonate groups, such as the salts of N-(2-aminoethyl)-β-alanine, of 2-(2-aminoethylamino)ethanesulphonic acid or of the addition product of IPDA and acrylic acid (EP-A 0 916 647, Example 1).

Mixtures of anionic or potentially anionic hydrophilicizing agents and nonionic hydrophilicizing agents can also be used.

A preferred embodiment for producing the specific polyurethane dispersions utilizes components A1) to A4) and B1) to B2) in the following amounts, the individual amounts always adding up to 100% by weight:

5% to 40% by weight of component A1),

55% to 90% by weight of A2),

0% to 20% by weight of the sum total of components A3) and B1)

0.1% to 25% by weight of the sum total of the components A4) and B2), with 0.1% to 10% by weight of anionic or potentially anionic hydrophilicizing agents from A4) and/or B2) being used, based on the total amounts of components A1) to A4) and B1) to B2).

A particularly preferred embodiment for producing the specific polyurethane dispersions utilizes components A1) to A4) and B1) to B2) in the following amounts, the individual amounts always adding up to 100% by weight:

5% to 35% by weight of component A1),

60% to 90% by weight of A2),

0.5% to 15% by weight of the sum total of components A3) and B1)

0.5% to 15% by weight of the sum total of components A4) and B2), with 0.5% to 7% by weight of anionic or potentially anionic hydrophilicizing agents from A4) and/or B2) being used, based on the total amounts of components A1) to A4) and B1) to B2).

A very particularly preferred embodiment for producing the specific polyurethane dispersions utilizes components A1) to A4) and B1) to B2) in the following amounts, the individual amounts always adding up to 100% by weight:

10% to 30% by weight of component Al),

65% to 85% by weight of A2),

0.5% to 14% by weight of the sum total of components A3) and B1)

1% to 10% by weight of the sum total of components A4) and B2), with 1% to 5% by weight of anionic or potentially anionic hydrophilicizing agents from A4) and/or B2) being used, and a proportion of 1% to 5% by weight, preferably less than 1.0% by weight and more preferably less than 0.5% by weight and most preferably no nonionically hydrophilicizing building blocks being used, based on the total amounts of components A1) to A4) and B1) to B2).

The production of the anionically hydrophilicized polyurethane dispersions (I) can be carried out in one or more stages in homogeneous phase or, in the case of a multistage reaction, partly in disperse phase. After completely or partially conducted polyaddition from A1) to A4) a dispersing, emulsifying or dissolving step is carried out. This is followed if appropriate by a further polyaddition or modification in disperse phase.

Any prior art process can be used, examples being the prepolymer mixing process, the acetone process or the melt dispersing process. The acetone process is preferred.

Production by the acetone process typically involves the constituents A2) to A4) and the polyisocyanate component A1) being wholly or partly introduced as an initial charge to produce an isocyanate-functional polyurethane prepolymer and optionally diluted with a water-miscible but isocyanate-inert solvent and heated to temperatures in the range from 50 to 120° C. The isocyanate addition reaction can be speeded using the catalysts known in polyurethane chemistry.

Useful solvents include the customary aliphatic, keto-functional solvents such as acetone, 2-butanone, which can be added not just at the start of the production process but also later, optionally in portions. Acetone and 2-butanone are preferred.

Other solvents such as xylene, toluene, cyclohexane, butyl acetate, methoxypropyl acetate, N-methylpyrrolidone, N-ethylpyrrolidone, solvents having ether or ester units can additionally be used and wholly or partly distilled off or in the case of N-methylpyrrolidone, N-ethylpyrrolidone remain completely in the dispersion. But preference is given to not using any other solvents apart from the aliphatic, keto-functional solvents mentioned.

Subsequently, any constituents of A1) to A4) not added at the start of the reaction are added.

In the production of the polyurethane prepolymer from A1) to A4), the amount of substance ratio of isocyanate groups to isocyanate-reactive groups is in the range from 1.05 to 3.5, preferably in the range from 1.2 to 3.0 and more preferably in the range from 1.3 to 2.5.

The reaction of components A1) to A4) to form the prepolymer is effected partially or completely, but preferably completely. Polyurethane prepolymers containing free isocyanate groups are obtained in this way, without a solvent or in solution.

The neutralizing step to effect partial or complete conversion of potentially anionic groups into anionic groups utilizes bases such as tertiary amines, for example trialkylamines having 1 to 12 and preferably 1 to 6 carbon atoms and more preferably 2 to 3 carbon atoms in every alkyl radical or alkali metal bases such as the corresponding hydroxides.

Examples thereof are trimethylamine, triethylamine, methyldiethylamine, tripropylamine, N-methylmorpholine, methyldiisopropylamine, ethyldiisopropylamine and diisopropylethylamine. The alkyl radicals may also bear for example hydroxyl groups, as in the case of the dialkylmonoalkanol-, alkyldialkanol- and trialkanolamines. Useful neutralizing agents further include if appropriate inorganic bases, such as aqueous ammonia solution, sodium hydroxide or potassium hydroxide.

Preference is given to ammonia, triethylamine, triethanolamine, dimethylethanolamine or diisopropylethylamine and also sodium hydroxide and potassium hydroxide, particular preference being given to sodium hydroxide and potassium hydroxide.

The bases are employed in an amount of substance which is between 50 and 125 mol % and preferably between 70 and 100 mol % of the amount of substance of the acid groups to be neutralized. Neutralization can also be effected at the same time as the dispersing step, by including the neutralizing agent in the water of dispersion.

Subsequently, in a further process step, if this has not already been done or only to some extent, the prepolymer obtained is dissolved with the aid of aliphatic ketones such as acetone or 2-butanone.

In the chain extension of stage B), NH₂− and/or NH-functional components are reacted, partially or completely, with the still remaining isocyanate groups of the prepolymer. Preferably, the chain extension/termination is carried out before dispersion in water.

Chain termination is typically carried out using amines B1) having an isocyanate-reactive group such as methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine or suitable substituted derivatives thereof, amide-amines formed from diprimary amines and monocarboxylic acids, monoketimes of diprimary amines, primary/tertiary amines, such as N,N-dimethylaminopropylamine.

When partial or complete chain extension is carried out using anionic or potentially anionic hydrophilicizing agents conforming to definition B2) with NH₂ or NH groups, chain extension of the prepolymers is preferably carried out before dispersion.

The aminic components B1) and B2) can optionally be used in water- or solvent-diluted form in the process of the present invention, individually or in mixtures, any order of addition being possible in principle.

When water or organic solvent is used as a diluent, the diluent content of the chain-extending component used in B) is preferably in the range from 70% to 95% by weight.

Dispersion is preferably carried out following chain extension. For dispersion, the dissolved and chain-extended polyurethane polymer is either introduced into the dispersing water, if appropriate by substantial shearing, such as vigorous stirring for example, or conversely the dispersing water is stirred into the chain-extended polyurethane polymer solutions. It is preferable to add the water to the dissolved chain-extended polyurethane polymer.

The solvent still present in the dispersions after the dispersing step is then typically removed by distillation. Removal during the dispersing step is likewise possible.

The residual level of organic solvents in the polyurethane dispersions (I) is typically less than 1.0% by weight and preferably less than 0.5% by weight, based on the entire dispersion.

The pH of the polyurethane dispersions (I) which are essential to the present invention is typically less than 9.0, preferably less than 8.5, more preferably less than 8.0 and most preferably is in the range from 6.0 to 7.5.

The solids content of the polyurethane dispersions (I) is preferably in the range from 35% to 70% by weight, more preferably in the range from 40% to 65% by weight, even more preferably in the range from 45% to 60% by weight and particularly in the range from 45% to 55% by weight.

The amount of anionic or potentially anionic groups on the particle surface, measured via an acid-base titration, is generally in the range from 2 to 500 mmol and preferably in the range from 30 to 400 mmol per 100 grams of solids.

The polyurethane dispersions (I) can be non-functional, or they can be in a functionalized state via hydroxyl or amino groups. In addition, the dispersions (I) may, in a non-preferred embodiment, also have reactive groups in the form of blocked isocyanate groups, as described in DE-A 19 856 412 for example.

The aqueous, cationically or potentially cationically hydrophilicized polyurethane dispersions (II) in the compositions essential to the present invention are obtainable by
C) isocyanate-functional prepolymers being produced from
   C1) organic polyisocyanates, as described in A1)
   C2) polymeric polyols mentioned in A2)
   C3) optionally hydroxyl-functional compounds, as described in A3), and
   C4) optionally isocyanate-reactive compounds that contain cationic groups or units convertible into cationic groups and/or optionally contain nonionically hydrophilicizing compounds mentioned in A4),
D) its free NCO groups then being wholly or partly reacted
   D1) optionally with the amino-functional compounds described in B1) and
   D2) optionally with isocyanate-reactive, preferably amino-functional, cationic or potentially cationic hydrophilicizing agents
      by chain extension, and the prepolymers being dispersed in water before, during or after step D), any potentially ionic groups present being converted into the ionic form by partial or complete reaction with a neutralizing agent.

Preferred aqueous cationic polyurethane dispersions (II) have a low degree of hydrophilic cationic groups, preferably from 2 to 200 milliequivalents and more preferably from 10 to 100 milliequivalents per 100 g of solid resin.

To achieve a cationic hydrophilicization, C4) and/or D2) shall use hydrophilicizing agents that have at least one NCO-reactive group and additionally contain cationic groups or units convertible into cationic groups. Examples of isocyanate-reactive groups are thiol and hydroxyl groups, primary or secondary amines are suitable any desired hydroxyl- and/or amino-functional mono-and particularly bifunctional compounds having at least one tertiary amine nitrogen atom, the tertiary nitrogen atoms of which can be at least partly converted into quaternary ammonium groups by neutralization or quaternization during or after the isocyanate polyaddition reaction. This includes for example compounds such as 2-(N,N-dimethylamino)ethylamine, N-methyl-diethanolamine, N-methyldiisopropanolamine, N-ethyldiethanolamine, N-ethyldiisopropanol-amine, N,N′-bis(2-hydroxyethyl)perhydropyrazine, N-methylbis(3-aminopropyl)amine, N-methyl-bis(2-aminoethyl)amine, N,N′-, N″-trimethyldiethylenetriamine, N,N-dimethylaminoethanol, N,N-diethylaminoethanol, 1-N,N-diethylamino-2-aminoethane, 1-N,N-diethylamino-3-aminopropane, 2-dimethylaminomethyl-2-methyl-1,3-propanediol, triethanolamine, tripropanolamine, triiso-propanolamine, N-isopropyldiethanolamine, N-butyldiethanolamine, N-isobutyldiethanolamine, N-oleyldiethanolamine, N-stearyldiethanolamine, ethoxylated cocoamine, N-allyldiethanolamine, N-methyldiisopropanolamine, N,N-propyldiisopropanolamine, N-butyldiisopropanolamine and/or N-cyclohexyldiisopropanolamine. The incorporation of tertiary and quaternary ammonium groups side by side or the incorporation of mixtures of the amino-functional hydrophilicizing agents mentioned is also possible.

To generate the cationic hydrophilicization, the ionic groups, i.e. the ternary or quaternary ammonium groups, are preferably incorporated using constructional components having tertiary amino groups by subsequent conversion of the tertiary amino groups into the corresponding ammonium groups through neutralization with organic or inorganic acids such as, for example phosphoric acid, hydrochloric acid, acetic acid, fumaric acid, adipic acid, maleic acid, lactic acid, tartaric acid, oxalic acid, malic acid, citric acid, ascorbic acid or N-methyl-N-(methyl-aminocarbonyl)aminomethanesulphonic acid, or through quaternization with suitable quaternizing agents such as, for example, methyl chloride, methyl iodide, dimethyl sulphate, benzyl chloride, ethyl chloroacetate or bromoacetamide. In principle this neutralization or quaternization of the constructional components comprising tertiary nitrogen can also be effected before or during the isocyanate polyaddition reaction, although this is less preferable. It is also possible to introduce ternary or quaternary ammonium groups into the polyisocyanate polyaddition products via polyether polyols used as and having tertiary amino groups by subsequent neutralization/quaternization of the tertiary amino groups. The incorporation of quaternary ammonium groups and of tertiary amino groups side by side or mixtures is also possible.

Neutralization can also be effected at the same time as the dispersing in water, for example by dissolving the neutralizing agent in water, concurrent addition of the neutralizing agent and of the water, or by addition of the neutralizing agent after the water has been added.

The degree of neutralization or quaternization as equivalents ratio of acid protons/quaternizing agent to potentially cationic groups in components C4 and/or D2 is generally set at between 20 and 300%, preferably 50 to 200% and more preferably between 70 and 130%.

The cationically hydrophilicized polyurethane dispersions (II) are prepared similarly to the principles and methods described for anionically hydrophilicized polyurethane dispersions (I).

The residual level of organic solvents in the polyurethane dispersions (II) is likewise typically less than 1.0% by weight and preferably less than 0.5% by weight, based on the entire dispersion.

The pH of the polyurethane dispersions (II) which are essential to the present invention is typically in the range from 2 to 8, preferably less than 7, more preferably less than 6 and most preferably in the range from 4 to 6.

A preferred embodiment for producing the cationically hydrophilicized polyurethane dispersions (II) utilizes the components C1) to C4) and D1) to D2) in the following amounts, the individual amounts always adding up to 100% by weight:

5% to 40% by weight of component C1),

55% to 90% by weight of component C2),

0.1% to 20% by weight of the sum total of components C3) and D1),

0.1% to 30% by weight of the sum total of components C4) and D2), with 0.1% to 20% by weight of cationic or potentially cationic hydrophilicizing agents from C4) and/or D2) being used, based on the total amounts of components C1) to C4) and DO to D2).

A particularly preferred embodiment for producing the cationically hydrophilicized polyurethane dispersions (II) utilizes the components C1) to C4) and D1) to D2) in the following amounts, the individual amounts always adding up to 100% by weight:

10% to 40% by weight of component C1),

65% to 90% by weight of component C2),

0.1% to 15% by weight of the sum total of components C3) and D1),

1% to 25% by weight of the sum total of components C4) and D2), with 1% to 15% by weight of cationic or potentially cationic hydrophilicizing agents from C4) and/or D2) being used, based on the total amounts of components C1) to C4) and D1) to D2).

A very particularly preferred embodiment for producing the cationically hydrophilicized polyurethane dispersions (II) utilizes the components C1) to C4) and D1) to D2) in the following amounts, the individual amounts always adding up to 100% by weight:

10% to 30% by weight of component C1),

65% to 85% by weight of component C2),

0.5% to 10% by weight of the sum total of components C3) and D1),

1% to 15% by weight of the sum total of components C4) and D2), with 1% to 10% by weight of cationic or potentially cationic hydrophilicizing agents from C4) and/or D2) being used, based on the total amounts of components C1) to C4) and D1) to D2).

The proportion of nonionically hydrophilicizing building blocks is preferably less than 20% by weight, more preferably less than 8% by weight and most preferably less than 3% by weight. In particular, no nonionically hydrophilicizing building blocks are present.

The solids content of the cationically hydrophilicized polyurethane dispersions (II) is generally in the range from 10% to 65% by weight, preferably in the range from 20% to 55% by weight and more preferably in the range from 25% to 45% by weight.

Preferred cationically hydrophilicized polyurethane dispersions (II) contain particles having a particle size of up to 800 nm, preferably in the range from 20 to 500 nm.

The amount of cationic or potentially cationic groups on the particle surface, measured via an acid-base titration, is generally between 2 to 500 mmol and preferably in the range from 30 to 400 mmol per 100 grams of solids.

As well as the polyurethane dispersions (I) and (II) it is also possible to use auxiliary and adjunct materials (III).

Examples of such auxiliary and adjunct materials (III) are foam auxiliaries such as foam formers and stabilizers, thickeners or thixotroping agents, antioxidants, light stabilizers, emulsifiers, plasticizers, pigments, fillers, pack stabilization additives, biocides, pH regulators, dispersions and/or flow control auxiliaries. Depending on the desired performance profile and intended purpose of the PU dispersion based coatings of the present invention, up to 70% by weight, based on total solids, of such fillers can be present in the end product.

Preferred auxiliary and adjunct materials (III) are foam auxiliaries such as foam formers and stabilizers. Useful foam auxiliaries include for example commercially available compounds such as fatty acid amides, sulphosuccinamides hydrocarbyl sulphonates, sulphates or fatty acid salts, wherein the lipophilic radical preferably contains 12 to 24 carbon atoms, and also alkylpolyglycosides obtainable in a conventional manner by reaction of comparatively long-chain monoalcohols (4 to 22 carbon atoms in the alkyl radical) with mono-, di- or polysaccharides (see for example Kirk-Othmer, Encyclopedia of Chemical Technology, John Wiley & Sons, Vol. 24, p. 29). Particularly suitable foam auxiliaries are EO-PO block copolymers obtainable in a conventional manner by addition of ethylene oxide and propylene oxide onto OH— or NH— functional starter molecules (see for example Kirk-Othmer, Encyclopedia of Chemical Technology, John Wiley & Sons, Vol. 24, p. 28). To improve foam formation, foam stability or the properties of the resulting polyurethane foam, still further additives may be included in component (III) as well as the EO-PO block copolymers. Such further additives can in principle be any known anionic, nonionic or cationic surfactant. Preferably, however, the EO-PO block copolymers are used alone as component (III).

Commercially available thickeners can be used, such as derivatives of dextrin, of starch, of polysaccharide such as guar gum or cellulose derivatives such as cellulose ethers or hydroxyethylcellulose, organic wholly synthetic thickeners based on polyacrylic acids, polyvinylpyrrolidones, poly(meth)acrylic compounds or polyurethanes (associative thickeners) and also inorganic thickeners, such as betonites or silicas.

It is likewise possible to add, incorporate or coat with antimicrobial or biological actives which have a positive effect, for example, in relation to wound healing and the avoidance of microbial loads.

Preferred actives of the aforementioned kind are those from the group of the antiseptics, growth factors, protease inhibitors and non-steroidal anti-inflammatories/opiates or else actives such as, for example, thrombin alfa for local blood coagulation.

In one preferred embodiment of the present invention, the active comprises at least a bacteriostat or a bactericide, most preferably an antiseptic biguanide and/or its salt, preferably the hydrochloride.

Biguanides are compounds derived from biguanide (C₂H₇N₅), particularly its polymers. Antiseptic biguanides are biguanides that have an antimicrobial effect, i.e. act as bacteriostats or preferably as bactericides. The compounds preferably have a broad action against many bacteria and can be characterized by a minimal microbicidal concentration (MMC, measured in the suspension test) of at least 0.5 μg/ml, preferably at least 12 or at least 25 μg/ml with regard to E. coli.

A preferred antiseptic biguanide according to this invention is poly(imino[iminocarbonyl]-iminopolymethylene), the use of poly(hexamethylene)biguanide (PHMB), also known as polyhexanide, as antiseptic biguanide being particularly preferred.

The term “antiseptic biguanides” according to this invention also comprehends metabolites and/or prodrugs of antiseptic biguanides. Antiseptic biguanides can be present as racemates or pure isoforms.

The foamed articles formed from polyurethane foams and the compositions according to the present invention preferably contain antiseptic biguanide and/or its salt, preferably the hydrochloride, in a concentration of from 0.010% to 20% by weight, very advantageously 0.1% to 5% by weight. The biguanide may have any desired molecular weight distribution.

In principle, although this is not preferable, the compositions that are essential to the present invention may also contain crosslinkers such as unblocked polyisocyanates, amide- and amine-formaldehyde resins, phenolic resins, polyaziridines, aldehydic and ketonic resins, for example phenol/formaldehyde resins, resols, furan resins, urea resins, carbamic ester resins, triazine resins, melamine resins, benzoguanamine resins, cyanamide resins or aniline resins.

Examples of compositions according to the present invention are recited hereinbelow, where the sum total of the weight % ages has a value of ≦100% by weight. These compositions, based on dry substance, typically comprise ≧80 parts by weight to ≦100 parts by weight of the dispersions (I) and (II) in total, ≧0 parts by weight to ≦10 parts by weight of foam auxiliary, ≧0 parts by weight to ≦10 parts by weight of crosslinker and ≧0 parts by weight to ≦10 parts by weight of thickener.

These compositions of the present invention, based on the dry substance, preferably comprise ≧85 parts by weight to ≦100 parts by weight of dispersions (I) and (II) in total, ≧0 parts by weight to ≦7 parts by weight of foam auxiliary, ≧0 parts by weight to ≦5 parts by weight of crosslinker, ≧0 parts by weight to ≦10 parts by weight of antiseptics or biocides and ≧0 parts by weight to ≦5 parts by weight of thickener.

These compositions of the present invention, based on the dry substance, more preferably comprise ≧89 parts by weight to ≦100 parts by weight of dispersions (I) and (II) in total, ≧0 parts by weight to ≦6 parts by weight of foam auxiliary, ≧0 parts by weight to ≦4 parts by weight of crosslinker and ≧0 parts by weight to ≦4 parts by weight of thickener.

The viscosity of component (I)/component (II) optionally blended with components (III) is preferably ≦1000 mPa·s, more preferably ≦700 mPa·s, even more preferably ≦500 mPa·s and particularly ≦200 mPa·s.

Examples of compositions according to the present invention which comprise ethylene oxide-propylene oxide block copolymers as foam stabilizers are recited hereinbelow. These compositions, based on dry substance, comprise ≧80 parts by weight to ≦100 parts by weight of dispersions (I) and (II) in total and ≧0 parts by weight to ≦20 parts by weight of the ethylene oxide-propylene oxide block copolymers. The compositions, based on dry substance, preferably comprise ≧85 parts by weight to ≦100 parts by weight of dispersions (I) and (II) in total and ≧0 to ≦15 parts by weight of the ethylene oxide-propylene oxide block copolymers. Particular preference is given to ≧90 parts by weight to ≦100 parts by weight of the dispersions (I) and (II) in total and ≧0 parts by weight to ≦10 parts by weight of the ethylene oxide-propylene oxide block copolymers and very particular preference is given to ≧94 parts by weight to ≦100 parts by weight of the dispersions (I) and (II) in total and ≧0 to ≦6 parts by weight of the ethylene oxide-propylene oxide block copolymers.

For the purposes of the present invention, “parts by weight” denotes a relative proportion, but not in the sense of % by weight. Consequently, the arithmetic sum total of the proportions by weight can also assume values below and above 100.

In addition to the recited components (I), (II) and (III), the compositions according to the present invention may also utilize further aqueous binders. Such aqueous binders can be constructed for example of polyester, polyacrylate, polyepoxy or other polyurethane polymers. Similarly, the combination with radiation-curable binders as described for example in EP-A-0 753 531 is also possible. It is further possible to also employ other anionic or nonionic dispersions, such as polyvinyl acetate, polyethylene, polystyrene, polybutadiene, polyvinyl chloride, polyacrylate or copolymer dispersions.

The microporous polyurethane foams are produced by the components (I), (II) and optionally (III) being mixed together and at the same time frothed. This homogenization can be effected for example by means of dynamic commixing or in a static mixer, preference being given to commixing by means of a static mixer.

Frothing in the process of the present invention is accomplished for example by mechanical stirring of the composition at high speeds of rotation, i.e. by inputting high shearing forces, or preferably by decompressing a blowing gas.

Useful blowing gases include in principle all blowing agents known per se to a person skilled in the art, for example hydrocarbons (for example propane, propene, n-butane, isobutane, butene, pentane), dimethyl ether, dimethoxymethane, carbon dioxide, nitrous oxide, nitrogen, oxygen, noble gases, air and less preferably hydrofluorocarbons (R134a for example) and chlorofluorocarbons (for example trichlorofluoromethane, dichlorodifluoromethane) and also mixtures thereof. Preference is given to using hydrocarbons, dimethyl ether or carbon dioxide, more preferably hydrocarbons and most preferably mixtures of C3 and C4 hydrocarbons.

The blowing gas (mixture) can be added to either of the two components (I) and (II) or else to both components. The addition of blowing gas can result in a changed viscosity for component (I) and/or (II). It is also conceivable to add different blowing gases to the two dispersions (I) and (II). Depending on the blowing gas, the result is a one- or two-phase water-gas mixture; preferably, the blowing agent dissolves completely in component (I) and/or (II).

Based on the aqueous overall mass of the dispersion (I) and/or (II) to be gassed, each of which optionally contains component (III), the amount of blowing agent added is preferably ≦30 parts by weight to ≧1 part by weight, more preferably ≦20 parts by weight to ≧1 part by weight and most preferably ≦10 parts by weight to ≧3 parts by weight.

Based on the resulting aqueous overall mass of the two dispersions (I) and (II) to be mixed with each other, each of which optionally contains component (III), the amount of dispersion (I) used is preferably ≧25 parts by weight to ≦90 parts by weight, more preferably ≧35 parts by weight to ≦80 parts by weight and most preferably ≧45 parts by weight to ≦75 parts by weight and the amount of dispersion (II) used is preferably ≦75 parts by weight to ≧10 parts by weight, more preferably ≦65 parts by weight to ≧20 parts by weight and most preferably ≦55 parts by weight to ≧25 parts by weight.

Based on the resulting overall volume of the two dispersions (I) and (II) to be mixed with each other, each of which optionally contains component (III), the amount of dispersion (I) used is preferably ≧25 parts by volume to ≦90 parts by volume, more preferably ≧35 parts by volume to ≦80 parts by volume and most preferably ≧45 parts by volume to ≦75 parts by volume and the amount of dispersion (II) used is preferably ≦75 parts by volume to ≧10 parts by volume, more preferably ≦65 parts by volume to ≧20 parts by volume and most preferably ≦55 parts by volume to ≧25 parts by volume.

It is preferably after <5 minutes, more preferably after <1 minute and most preferably after <30 seconds that consolidation of the foam paste occurs. It is preferably not until after 1 second, more preferably not until after 3 seconds and most preferably not until after 5 seconds that consolidation occurs in order that uniform distribution or spreading of the foam paste may initially be made possible.

A satisfactory rate of drying of the foams is observed, in a preferred embodiment, at a temperature as low as 20° C., so that drying on injured or uninjured human or animal tissue is possible without problems. However, temperatures above 30° C. may also be used for faster drying of the foams. However, drying temperatures should not exceed 200° C., preferably 160° C. and more preferably 140° C., since undesirable yellowing of the foams is just one problem that can otherwise occur. Drying in two or more stages is also possible. It is further possible to dry using infrared radiation or microwave radiation. Drying on human or animal tissue is preferably done without heating through an external supply of heat.

Before drying, the foam densities of the polyurethane foams are typically in the range from 50 to 800 g/litre, preferably in the range from 100 to 700 g/litre and more preferably in the range from 200 to 600 g/litre (mass of all input materials [in g] based on the foam volume of one litre). The density of the dried foams is typically in the range from 50 to 800 g/litre, preferably in the range from 100 to 600 g/litre and most preferably in the range from 200 to 500 g/litre.

The polyurethane foams can be produced in virtually any desired thickenss. Dried foams are typically produced to have a thickness in the range from 0.1 mm to 100 mm, preferably in the range from 1 mm to 30 mm, more preferably in the range from 5 mm to 20 mm and most preferably in the range from 5 to 10 mm. Applying two or more layers of the polyurethane foams in succession is also possible, with or without application of interlayers which do not correspond to the polyurethane foams of the present invention.

Irrespectively of the method of drying used, the foams of the present invention consolidate within a few seconds from application to obtain a microporous, at least partly open-pore structure having intercommunicating cells. The polyurethane foams have good mechanical strength and elasticity even whilst still in the moist state. Maximum elongation is typically greater than 50% and preferably greater than 100% (determination to DIN 53504).

The resulting, still moist foams have a pH of ≧4 to ≦9, preferably of ≧5 to ≦7 and more preferably of ≧5 to ≦6.

Although these foams already contain water due to the dispersion from which they were formed, they are still capable of absorbing an additional volume of liquid. No significant swelling of the hydrophilic foams occurs.

The polyurethane foams of the present invention experience a volume shrinkage during drying that is less than 30%, preferably less than 20% and more preferably less than 10% based on the foam volume immediately after the foaming operation.

The water vapour transmission rate of the still moist foams is typically in the range from 1000 to 8000 g/24 h*m2, preferably 2000 to 8000 g/24 h*m2 and more preferably 3000 to 8000 g/24 h*m2 (determined to DIN EN 13726-2 Part 3.2).

Exposed in their dried state to isotonic sodium chloride solution at 23° C., the polyurethane foams of the present invention swell less than 5% and preferably less than 2%.

The foams of the present invention can also be applied to substrates. Suitable substrates for this include for example textile fabrics, sheetlike substrates composed of metal, glass, ceramic, concrete, natural stone, leather, natural fibres and plastics such as PVC, polyolefins, polyurethane or the like. Textile fabrics herein are to be understood as meaning, for example, wovens, knits, bonded and unbonded fibrous nonwoven webs. The textile fabrics can be constructed of synthetic, natural fibres and/or mixtures thereof. In principle, textiles composed of any desired fibres are suitable for the process of the present invention.

Suitable substrates also include, in particular, papers or foils or self-supporting films that facilitate simple detachment of the wound contact material before its use for covering an injured site. Human or animal tissue such as skin can similarly serve as a substrate, so that direct closure of an injured site by an in-situ prepared wound contact material is possible.

The polyurethane foams can moreover be adhered, laminated or coated to or with further materials, for example materials based on hydrogels, (semi)permeable foils or self-supporting film, coatings or other foams.

It is also possible for wounds or uninjured parts of the body to be initially covered with a foil, a self-supporting film or a piece of paper and then the foam of the present invention to be cured without wound contact. This would make it possible to avoid direct wound contact during curing. It is likewise possible to remove the release foil or film or the paper from the wound or uninjured part of the body after curing, so that direct wound contact with the foam is engendered after the cure.

It is also possible for the foam to be expanded in a mould after commixing but before complete curing and for the moulding parts to be removed after curing. This makes it possible to produce three-dimensionally shaped foams in a controlled manner, for example in order that they may be conformed to a wound or else to an injured or uninjured part of the body such as the heel for example.

### EXAMPLES

Unless indicated otherwise, all percentages are by weight.

Solids contents were determined in accordance with DIN-EN ISO 3251.

NCO contents were, unless expressly mentioned otherwise, determined volumetrically in accordance with DIN-EN ISO 11909.

The reported viscosities were determined by means of rotary viscometry to DIN 53019 at 23° C. using a rotary viscometer from Anton Paar Germany GmbH, Ostfildern, DE.

### Charge Determination

A portion of the sample is weighed out to an accuracy of 0.0001 g (mass typically between 0.2 g and 1 g, depending on amount of charge), admixed with a 5% by weight aqueous surfactant solution (Brij-96 V, Fluka, Buchs, Switzerland product No. 16011) and doubly deionized water and, following addition of a defined amount of hydrochloric acid (0.1 N in order that the batch may have an initial pH of approximately pH 3; KMF Laborchemie GmbH, Lohmar, Art. No.: KMF.01-044.1000), titrated with aqueous sodium hydroxide standard solution (0.05 N; Bernd Kraft GmbH, Duisburg, Art. No.: 01056.3000). In addition, in order to differentiate between the surface charge and the liquid-phase charge, a portion (approximately 30 g) of the dispersion is treated with Lewatit VP-OC 1293 ion exchanger (employing the 10-fold exchange capacity relative to the total charge determined, stirring time 2.0 h, Lanxess AG, Leverkusen, mixed anion/cation exchanger) and the resulting dispersion, after filtration (E-D fast sieve, cotton fabric 240 μm from Erich Drehkopf GmbH, Ammersbek). The surface charge is determined in the titration of the sample after ion exchanger treatment. By calculating the difference to the total charge it is possible to determine the liquid-phase charge.

The determination of the surface charge from the points of equivalence provides, within the bounds of measurement accuracy, a comparable value to the determination of basic groups from the minimum consumption of sodium hydroxide solution, relative to the amount of hydrochloric acid added.

### Substances and Abbreviations Used

Diaminosulphonate: NH₂—CH₂CH₂—NH—CH₂CH₂—SO₃Na (45% in water)
Desmophen® C2200: polycarbonate polyol, OH number 56 mg KOH/g, number average molecular weight 2000 g/mol (BayerMaterialScience AG, Leverkusen, DE)
PolyTHF® 2000: polytetramethylene glycol polyol, OH number 56 mg KOH/g, number average molecular weight 2000 g/mol (BASF AG, Ludwigshafen, DE)
PolyTHF® 1000: polytetramethylene glycol polyol, OH number 112 mg KOH/g, number average molecular weight 1000 g/mol (BASF AG, Ludwigshafen, DE)
Polyether LB 25: monofunctional polyether based on ethylene oxide/propylene oxide, number average molecular weight 2250 g/mol, OH number 25 mg KOH/g (Bayer Material Science AG, Leverkusen, DE)
Pluronic® PE3500: ethylene oxide-propylene oxide block copolymer, ethylene oxide fraction about 50%, number average molecular weight about 1900 g/mol (BASF AG, Ludwigshafen, DE)
Pluronic® PE6800: ethylene oxide-propylene oxide block copolymer, ethylene oxide fraction about 80%, number average molecular weight about 8000 g/mol (BASF AG, Ludwigshafen, DE)

### Example 1: Polyurethane Dispersion (I)

450 g of PolyTHF® 1000 and 2100 g of PolyTHF® 2000 were heated to 70° C. Then, a mixture of 225.8 g of hexamethylene diisocyanate and 298.4 g of isophorone diisocyanate was added at 70° C. during 5 min, followed by stirring at 100-115° C. until the NCO value had dropped below the theoretical NCO value. The ready-produced prepolymer was dissolved with 5460 g of acetone at 50° C. and subsequently admixed with a solution of 29.5 g of ethylenediamine, 143.2 g of diaminosulphonate and 610 g of water by metered addition during 10 min. The resulting mixture was subsequently stirred for 15 min. Then, a dispersion was formed during 10 min by addition of 1880 g of water. This was followed by removal of the solvent by distillation in vacuo (in the course of which water additionally distilled off was replaced) to obtain a storage-stable dispersion having the following properties:
Solids content: 56%
Particle size (LCS): 276 nm
pH (23° C.): 7.0
Viscosity: 1000 mPas

### Example 2: Polyurethane Dispersion (I)

1165.5 g of PolyTHF® 2000, 250.3 g of PoIyTHF® 1000 and 14.3 g of Polyether LB 25 polyether were heated to 70° C. in a standard stirred apparatus. Then, a mixture of 158.0 g of hexamethylene diisocyanate and 208.8 g of isophorone diisocyanate was added at 70° C. during 5 min, followed by stirring at 120° C. until the NCO value had dropped slightly below the theoretical NCO value. The ready-produced prepolymer was dissolved with 4620 g of acetone and cooled to 50° C. in the process and subsequently admixed with a solution of 16.8 g of ethylenediamine, 68.7 g of isophoronediamine, 63.5 g of diaminosulphonate and 730 g of water by metered addition during 10 min The resulting mixture was subsequently stirred for 10 min. Then, a dispersion was formed by addition of 383 g of water. This was followed by removal of the solvent by distillation in vacuo to obtain a storage-stable dispersion having the following properties:
Solids content: 58.1%
Particle size (LCS): 446 nm
pH (23° C.): 7.2
Viscosity (23° C.): 331 mPas

### Example 3: Polyurethane Dispersion (I)

467.5 g of an OH-functional polyester formed from adipic acid, hexanediol and neopentyl glycol and having an average molecular weight of 1700 g/mol were heated to 65° C. Then, a mixture of 54.7 g of isophorone diisocyanate and 64.6 g of methylenebis(4-isocyanatocyclohexane) was added at 70° C. during 5 min, followed by stirring at 100-115° C. until the NCO value had dropped to below the theoretical NCO value. The ready-produced prepolymer was dissolved with 1040 g of acetone at 50° C. and subsequently mixed with 7.9 g of methylenebis(4-aminocyclohexane) and after a further 5 minutes with a solution of 1.0 g of ethylenediamine, 41.8 g of diaminosulphonate and 180 g of water added by metered addition during 3 min. The resulting mixture was subsequently stirred for 5 min. Thereafter, a dispersion was formed during 10 min by addition of 241 g of water. This was followed by removal of the solvent by distillation in vacuo and addition of a further 370 g of water to obtain a storage-stable dispersion having the following properties:
Solids content: 43%
Particle size (LCS): 113 nm
pH: 7.2
Viscosity: 410 mPas

### Example 4: Polyurethane Dispersion (II)

882.3 g of Desmophen C 2200 and 189.1 g of PolyTHF® 1000 were heated to 70° C. in a standard stirred apparatus. Then, a mixture of 143 g of hexamethylene diisocyanate and 189 g of isophorone diisocyanate was added at 70° C. during 5 min and the resulting mixture was stirred at 120° C. until the NCO value had dropped slightly below the theoretical NCO value. The prepolymer was cooled down to 80° C. and then admixed with 95.4 g of N-methyldiethanolamine. The resulting mixture was stirred at 80° C. for a further 30 minutes and then dissolved with 1500 g of acetone and cooled to 50° C. in the process. Then, a solution of 17.1 g of isophoronediamine in 31 g of acetone and after a further 10 minutes 0.5 g of ethylenediamine in 2.3 g of water were metered in during 10 min. The resulting mixture was subsequently stirred for 10 min. Then, the mixture was neutralized by addition of 86.9 g of 85% aqueous phosphoric acid and directly thereafter dispersed with 3450 g of water. This was followed by the removal of the solvent by distillation in vacuo to obtain a storage-stable dispersion.

The polyurethane dispersion obtained had the following properties:
Solids content: 31.4%
Particle size (LCS): 87 nm
pH (23° C.): 4.4
Viscosity (23° C.): 130 mPas

### Example 5: Polyurethane Dispersion (II)

185.2 g of Desmophen C 2200 and 39.7 g of Po1yTHF® 1000 were heated to 70° C. in a standard stirred apparatus. Then, a mixture of 30.0 g of hexamethylene diisocyanate and 39.6 g of isophorone diisocyanate was added at 70° C. during 5 min and the resulting mixture was stirred at 120° C. until the NCO value had dropped slightly below the theoretical NCO value. The prepolymer was cooled down to 80° C. and then admixed with 20.0 g of N-methyldiethanolamine. The resulting mixture was stirred at 80° C. for a further 30 minutes and then dissolved with 315 g of acetone and cooled to 50° C. in the process. Then, a solution of 3.6 g of isophoronediamine in 6.4 g of acetone and after a further 10 minutes 0.1 g of ethylenediamine in 0.5 g of water were metered in during 10 min. The resulting mixture was subsequently stirred for 10 min. Then, the mixture was neutralized by addition of 143.4 g of 1N aqueous hydrochloric acid and directly thereafter dispersed with 600 g of water. This was followed by the removal of the solvent by distillation in vacuo to obtain a storage-stable dispersion having the following properties:
Solids content: 29.6%
Particle size (LCS): 66 nm
pH (23° C.): 5.8
Viscosity (23° C.): 30 mPas

### Example 6: Polyurethane Dispersion (II)

185.2 g of PolyTHF® 2000 and 39.7 g of PolyTHF® 1000 were heated to 70° C. in a standard stirred apparatus. Then, a mixture of 30.0 g of hexamethylene diisocyanate and 39.6 g of isophorone diisocyanate was added at 70° C. during 5 min and the resulting mixture was stirred at 120° C. until the NCO value had dropped slightly below the theoretical NCO value. The prepolymer was cooled down to 80° C. and then admixed with 20.0 g of N-methyldiethanolamine. The resulting mixture was stirred at 80° C. for a further 30 minutes and then dissolved with 315 g of acetone and cooled to 50° C. in the process. Then, a solution of 3.6 g of isophoronediamine in 6.4 g of acetone and after a further 10 minutes 0.1 g of ethylenediamine in 0.5 g of water were metered in during 10 min. The resulting mixture was subsequently stirred for 10 min. Then, the mixture was neutralized by addition of 143.4 g of 1N aqueous hydrochloric acid and directly thereafter dispersed with 600 g of water. This was followed by the removal of the solvent by distillation in vacuo to obtain a dispersion having the following properties.
Solids content: 31.0%
Particle size (LCS): 180 nm
pH (23° C.): 6.1
Viscosity (23° C.): 25 mPas

### Example 7: Coagulation of Two Dispersions

400 g of a dispersion according to Example 1 were admixed with 6 g of Pluronic® PE6800 and 118 g of water. Then, one of the two chambers of a commercially available spray can featuring two-chamber aerosol technology was filled with 30 g of the anionic dispersion mixture, while the other chamber was filled with 30 g of a cationic dispersion according to Example 4. The can was pregassed with about 3 g of a propane-butane mixture as blowing agent. In addition, the chamber containing the anionic dispersion mixture was filled with about 1.5 g of the same blowing gas and the can was subsequently sealed. After one day of storage at room temperature, the two components, mixed by means of a static mixer, were spray dispensed. The mixing ratio of the two components relative to each other was determined as 2.1 g of anionic PUD mixture to 1.0 g of cationic PUD mixture.

The resulting foam consolidated within a few seconds after spraying without any evolution of heat (in the form of a temperature increase) being observed. The fresh white foam obtained had whilst still in the moist state an absorbence of 130% by weight (determined to DIN EN 13726-1 Part 3.2) and also a water vapour transmission rate of 4000 g/24 h*m² at a foam thickness of about 3-4 mm (determined to DIN EN 13726-2 Part 3.2). It did not display any noticeable swelling even after complete drying and renewed absorption of liquid.

### Example 8: Coagulation of Two Dispersions

One of the two chambers of a commercially available spray can featuring two-chamber aerosol technology was filled with 30 g of an anionic dispersion according to Example 2, while the other chamber was filled with 30 g of a cationic dispersion mixture according to Example 5. The can was pregassed with about 3 g of a propane-butane mixture as blowing agent. In addition, the chamber containing the anionic dispersion mixture was filled with about 1.5 g of the same blowing gas and the can was subsequently sealed. After one day of storage at room temperature, the two components, mixed by means of a static mixer, were spray dispensed. The mixing ratio of the two components relative to each other was determined as 1.0 g of anionic PUD mixture to 1.0 g of cationic PUD mixture.

The resulting foam consolidated within a few seconds after spraying without any evolution of heat being observed. The fresh white foam obtained had whilst still in the moist state a noticeable absorbence of isotonic sodium chloride solution and also a good water vapour permeability. It did not display any noticeable swelling even after complete drying and renewed absorption of liquid.

### Example 9: Coagulation of Two Dispersions

400 g of a dispersion according to Example 6 were admixed with 1.2 g of Pluronic® PE3500. Then, one of the two chambers of a commercially available spray can featuring two-chamber aerosol technology was filled with 30 g of this dispersion mixture, while the other chamber was filled with 30 g of a dispersion according to Example 3. The can was pregassed with about 3 g of a propane-butane mixture as blowing agent. In addition, the chamber containing the anionic dispersion mixture was filled with about 1.5 g of the same blowing gas and the can was subsequently sealed. After one day of storage at room temperature, the two components, mixed by means of a static mixer, were spray dispensed. The mixing ratio found for the two components relative to each other was 1.2:1 (anionic PUD mixture:cationic PUD mixture).

The resulting foam consolidated within a few seconds after spraying without any evolution of heat being observed. The fresh white foam obtained had whilst still in the moist state a noticeable saline absorbence and also a water vapour transmission rate of 4600 g/24 h*m² at a foam thickness of about 3-4 mm (determined to DIN EN 13726-2 Part 3.2). It did not display any noticeable swelling even after complete drying and renewed absorption of liquid.

1.-15. (canceled)

16. A composition obtained by mixing an aqueous anionically hydrophilicized polyurethane dispersion and an aqueous cationically or potentially cationically hydrophilicized polyurethane dispersion.

17. The composition according to claim 16, wherein the aqueous anionically hydrophilicized polyurethane dispersions are obtained by
A) providing isocyanate-functional prepolymers produced from
A1) organic polyisocyanates
A2) polymeric polyols having number average molecular weights of from 400 to 8000 g/mol, and OH functionalities in the range from 1.5 to 6, and
A3) optionally hydroxyl-functional compounds having molecular weights of from 62 to 399 g/mol, and
A4) optionally isocyanate-reactive, anionic or potentially anionic and/or optionally nonionic hydrophilicizing agents,
B) wholly or partly reacting free NCO groups of the isocyanate-functional prepolymers by chain extension
B1) optionally with amino-functional compounds having molecular weights of from 32 to 400 g/mol and
B2) optionally with isocyanate-reactive, anionic or potentially anionic hydrophilicizing agents
wherein the isocyanate-functional prepolymers are dispersed in water before, during or after B), wherein any potentially ionic groups present are converted into the ionic form by partial or complete reaction with a neutralizing agent.

18. The composition according to claim 16, wherein the aqueous cationically or potentially cationically hydrophilicized polyurethane dispersions are obtained by
C) providing isocyanate-functional prepolymers produced from
C1) organic polyisocyanates,
C2) polymeric polyols,
C3) optionally hydroxyl-functional compounds, and
C4) optionally isocyanate-reactive compounds comprising cationic groups or units convertible into cationic groups and/or optionally nonionically hydrophilicizing compounds,
D) wholly or partly reacting free NCO groups of the isocyanate-functional prepolymers by chain extension
D1) optionally with the amino-functional compounds described in B1) and
D2) optionally with isocyanate-reactive, cationic or potentially cationic hydrophilicizing agents
wherein the isocyanate-functional prepolymers are dispersed in water before, during or after D), any potentially ionic groups are converted into the ionic form by partial or complete reaction with a neutralizing agent.

19. The composition according to claim 16, wherein each of the aqueous polyurethane dispersions have an ionic group content of from 2 to 200 milliequivalents per 100 g of solid resin in the respective dispersion.

20. The composition according to claim 16, wherein the polymeric polyols A2) comprise polytetramethylene glycol polyols.

21. The composition according to claim 20, wherein the polymeric polyols A2) comprise a mixture of polycarbonate polyols and polytetramethylene glycol polyols wherein the proportion of polycarbonate polyols in the mixture is from 0% to 80% by weight and the proportion of polytetramethylene glycol polyols in the mixture is from 100% to 20% by weight.

22. The composition according to claim 18, wherein the polymeric polyols C2) comprise polytetramethylene glycol polyols.

23. The composition according to claim 22, wherein the polymeric polyols C2) comprise a mixture of polycarbonate polyols and polytetramethylene glycol polyols wherein the proportion of polycarbonate polyols in the mixture is from 0% to 80% by weight and the proportion of polytetramethylene glycol polyols in the mixture is from 100% to 20% by weight.

24. The composition according to claim 16, wherein the organic polyisocyanates A1) comprise polyisocyanates or polyisocyanate mixtures having exclusively aliphatically and/or cycloaliphatically attached isocyanate groups and an average NCO functionality of from 2 to 4 for the polyisocyanate mixture.

25. The composition according to claim 24, wherein the organic polyisocyanates comprise polyisocyanates or polyisocyanate mixtures having exclusively aliphatically and/or cycloaliphatically attached isocyanate groups and an average NCO functionality of from 2 to 4 for the polyisocyanate mixture.

26. The composition according to claims 16, wherein the composition further comprises auxiliary and/or adjunct materials.

27. A process for producing polyurethane foams comprising providing the composition according to claim 16 and frothing the composition during or after the complete mixing of the individual components to form a foam and curing the foam.

28. The process according to claim 27, wherein the curing comprises a consolidation of the foam and a subsequent drying step of the foam.

29. A polyurethane foam obtained by the process according to claim 27.

30. The polyurethane foam according to claim 29 having a density in the consolidated and dried state of from 50 to 800 g/litre.

31. The polyurethane foam according to claim 29 having a water vapour transmission rate of from 1000 to 8000 g/24 h*m² as determined by DIN EN 13726-2 Part 3.2.

32. The polyurethane foams according to claim 29, wherein during drying the polyurethane foam, on average, undergoes a volume shrinkage of less than 30% based on the foam volume immediately after the foaming operation.

33. A wound contact material comprising the polyurethane foam according to claim 29.

34. A dressing material comprising the polyurethane foam according to claim 29.
